(19) United States
(12) Patent Application Publication
(10) Pub. No.: US 2025/0032408 A1
(43) Pub. Date: Jan. 30, 2025

(54) REACTIVE POLYMERS AND HYDROGELS FORMED FROM THE SAME

(71) Applicant: Boston Scientific Scimed, Inc., Maple Grove, MN (US)

(72) Inventors: Joseph Thomas Delaney, JR., Minneapolis, MN (US); Cristian Parisi, Boston, MA (US); Yen-Hao Hsu, Shrewsbury, MA (US); Rick Dorn, Plymouth, MN (US)

(73) Assignee: Boston Scientific Scimed, Inc., Maple Grove, MN (US)

(21) Appl. No.: 18/781,571

(22) Filed: Jul. 23, 2024

Related U.S. Application Data
(60) Provisional application No. 63/515,523, filed on Jul. 25, 2023.

(51) Int. Cl. A61K 9/06; A61K 47/34; A61L 27/52; C08G 65/332; C08G 65/333

(52) U.S. Cl. CPC A61K 9/06; A61K 47/34; A61L 27/52; C08G 65/3328; C08G 65/33396; A61L 2400/06

(57) ABSTRACT

In some aspects, the present disclosure provides reactive polymers that comprise one or more hydrophilic polymer segments having a plurality of hydrophilic polymer segment ends and a plurality of reactive moieties covalently linked to at least a portion of the hydrophilic polymer segment ends. The reactive moieties are 3,4-substituted-2,5-pyrrolidinedione moieties in which the 3-carbon and the 4-carbon form part of at least one ring in addition to the 2,5-pyrrolidinedione ring, and the 2,5-pyrrolidinedione ring nitrogen atom of each of the 3,4-substituted-2,5-pyrrolidinedione moieties is linked to one of the hydrophilic polymer segment ends. In other aspects, the present disclosure provides systems for forming hydrogel compositions and methods of treatment that employ such reactive polymers and methods of synthesizing such reactive polymers.

### CROSS REFERENCE TO RELATED APPLICATIONS

This application claims the benefit of U.S. Provisional Patent Application Ser. No. 63/515,523 filed on Jul. 25, 2023, the disclosure of which is incorporated herein by reference.

### FIELD

The present disclosure relates to reactive polymers, to crosslinked hydrogels formed from such reactive polymers, and to methods of making and using such crosslinked hydrogels, among other aspects. The crosslinked hydrogels of the present disclosure are useful, for example, in various medical applications.

### BACKGROUND

Currently, there are several commercialized hydrogel formulations that form through the chemical crosslinking of a hydrophilic star poly(ethylene glycol) (PEG) polymer functionalized with activated ester end groups, specifically, succinimidyl glutarate end groups, which react with a polyamine functionalized crosslinker, specifically trilysine, to form peptide crosslinks.

To manufacture the succinimidyl glutarate end groups, the available hydroxy-terminated end groups of the star PEG polymer are functionalized with glutaric anhydride, and then allowed to react with N-hydroxysuccinimide in the presence of a dicyclohexylcarbodiimide coupling agent to yield the reactive succinimidyl glutarate end groups.

This process, however, has its limitations. With reference now to FIG. 1, during the final synthetic step, there is a competing side reaction that may take place, whereby N-hydroxysuccinimide 112 condenses and undergoes a Lossen rearrangement in the presence of dicyclohexylcarbodiimide 114, forming an impurity, N-succinimidoxycarbonyl-β-alanine N-succinimidyl ester 116 (CAS #21994-89-8, hereinafter referred to as the β-alanine impurity).

One issue with the β-alanine impurity is that the β-alanine impurity is also an activated ester and will compete with the reactive star polymer to crosslink with the trilysine. With the β-alanine impurity taking up space on the trilysine crosslinker, the maximum achievable crosslink density will always be lower than intended. This can impact important performance characteristics such as gel time, in vivo persistence, mechanical properties, swellability, and by extension, radiopacity over time.

For this reason, it is desirable to have an alternative activated ester formation strategy that yields a product that will react with amine groups to form peptide crosslinks, but will not form the β-alanine impurity.

### SUMMARY

In some aspects, the present disclosure provides reactive polymers that comprise one or more hydrophilic polymer segments having a plurality of hydrophilic polymer segment ends and a plurality of reactive moieties covalently linked to at least a portion of the hydrophilic polymer segment ends. The reactive moieties are 3,4-substituted-2,5-pyrrolidinedione moieties in which the 3-carbon and the 4-carbon form part of at least one ring in addition to the 2,5-pyrrolidinedione ring, and the 2,5-pyrrolidinedione ring nitrogen atom of each of the 3,4-substituted-2,5-pyrrolidinedione moieties is linked to one of the hydrophilic polymer segment ends.

In some embodiments, the 2,5-pyrrolidinedione ring nitrogen atom is linked to the one of the hydrophilic polymer segment ends through a hydrolysable ester group.

In some embodiments, which can employed be in conjunction with the above aspects and embodiments, the one or more hydrophilic polymer segments comprise a single hydrophilic polymer segment, and the plurality of hydrophilic polymer segment ends comprise first and second ends of the single hydrophilic polymer segment.

In some embodiments, which can employed be in conjunction with the above aspects and embodiments, the reactive polymers comprise a plurality of polymer arms linked to a core region, where at least a portion the polymer arms each comprise one of the plurality hydrophilic polymer segments and one of the plurality of reactive moieties. In some of these embodiments, a first end of the one of the plurality of hydrophilic polymer segments is covalently linked to the core region, a second end of the one of the plurality of hydrophilic polymer segment is covalently linked to a first end of a cyclic anhydride residue, and the one of the plurality of reactive moieties is covalently linked to a second end of the cyclic anhydride residue.

In some embodiments, which can employed be in conjunction with the above aspects and embodiments, the hydrophilic polymer segment comprises one or more monomer residues selected from ethylene oxide, propylene oxide, N-vinyl pyrrolidone and oxazoline monomer residues.

In some embodiments, which can employed be in conjunction with the above aspects and embodiments, each of the reactive moieties comprise the following formula,

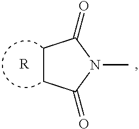

where R denotes a single-ring or a multi-ring structure. In some of these embodiments, wherein R denotes a cyclopentane structure, a cyclopentene structure, a cyclohexane structure, a cyclohexene structure, a norbornane structure, a norbornene structure, a 7-oxonorbornane structure, or a 7-oxonorbornene structure.

In some aspects, the present disclosure provides systems for forming hydrogel compositions that comprises (a) a nucleophilic compound and (b) a reactive polymer in accordance with any of the above aspects and embodiments.

In some embodiments, the nucleophilic compound is a polyamino compound.

In some embodiments, which can employed be in conjunction with the above aspects and embodiments, the systems comprise a first composition that comprises the nucleophilic compound and a second composition that comprises the reactive polymer. In some of these embodiments, the systems further comprise an accelerant composition.

In some embodiments, which can employed be in conjunction with the above aspects and embodiments, the systems comprise one or more additional agents selected from therapeutic agents, imaging agents, colorants, tonicity adjusting agents, suspension agents, wetting agents, and pH adjusting agents.

In some embodiments, which can employed be in conjunction with the above aspects and embodiments, the systems further comprise a delivery device.

In some aspects, the present disclosure provides methods of treatment comprising administering to a subject a mixture that comprises (a) a nucleophilic compound and (b) a reactive polymer in accordance with any of the above aspects and embodiments, wherein the mixture is administered under conditions such that the nucleophilic compound and the reactive polymer crosslink after administration.

In some aspects, the present disclosure provides synthesis methods that comprise condensing (a) a 3,4-substituted-1-hydroxy-2,5-pyrrolidinedione compound in which the 3-carbon and the 4-carbon form part of at least one ring in addition to the 2,5-pyrrolidinedione ring with (b) a precursor polymer comprising one or more hydrophilic polymer segments having a plurality of hydrophilic polymer segment ends that are terminated in carboxylic acid groups, thereby forming a reactive polymer comprising a plurality of reactive moieties covalently linked to the hydrophilic polymer segment ends, wherein each of the reactive moieties is a 3,4-substituted-2,5-pyrrolidinedione moiety in which the 3-carbon and the 4-carbon form part of at least one ring in addition to the 2,5-pyrrolidinedione ring, and wherein the 2,5-pyrrolidinedione ring nitrogen atom of the 3,4-substituted-2,5-pyrrolidinedione moiety is linked to one of the hydrophilic polymer segment ends.

In some embodiments, the precursor polymer is formed by reacting an acid anhydride with a polymer comprising one or more hydrophilic polymer segments having a plurality of hydrophilic polymer segment ends that are terminated in hydroxyl groups.

In some embodiments, which can employed be in conjunction with the above aspects and embodiments, the one or more hydrophilic polymer segments of the precursor polymer comprise a single hydrophilic polymer segment, and the plurality of hydrophilic polymer segment ends comprise first and second ends of the single hydrophilic polymer segment.

In some embodiments, which can employed be in conjunction with the above aspects and embodiments, the precursor polymer comprises a plurality of polymer arms linked to a core region, at least a portion the polymer arms each comprising one of the plurality hydrophilic polymer segments and one of the plurality of reactive moieties.

In some embodiments, which can employed be in conjunction with the above aspects and embodiments, the 3,4-substituted-1-hydroxy-2,5-pyrrolidinedione compound is a compound of the following formula,

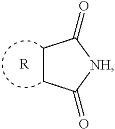

where R denotes a single-ring or a multi-ring structure. In some of these embodiments, R denotes a cyclopentane structure, a cyclopentene structure, a cyclohexane structure, a cyclohexene structure, a norbornane structure, a norbornene structure, a 7-oxonorbornane structure or a 7-oxonorbornene structure.

Potential benefits associated with the present disclosure include improvement of one or more of the following properties: radiocontrast, in vivo persistence, mechanical properties, swellability and cure kinetics.

The above and other aspects, embodiments, features and benefits of the present disclosure will be readily apparent from the following detailed description.

### BRIEF DESCRIPTION OF THE DRAWINGS

FIG. 1 schematically illustrates the formation of the β-alanine impurity via a Lossen rearrangement of N-hydroxysuccinimide.

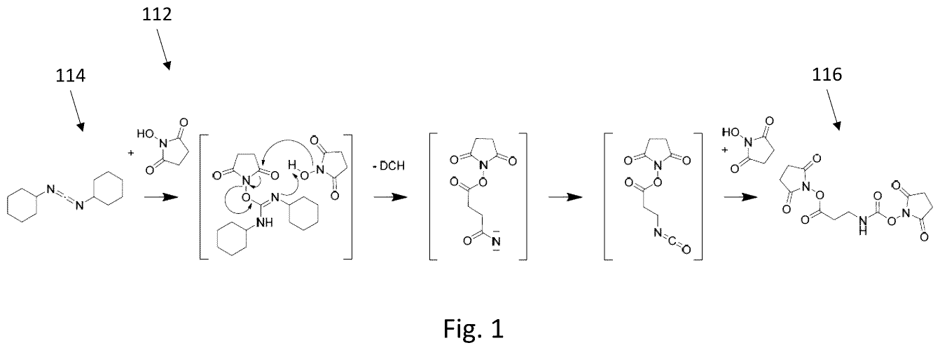

FIG. 2 schematically illustrates a method for forming a reactive multi-arm polymer, according to an aspect of the present disclosure.

FIG. 3 illustrates a delivery device, in accordance with an embodiment of the present disclosure.

FIG. 4 illustrates a delivery device, in accordance with another embodiment of the present disclosure.

### DETAILED DESCRIPTION

In some aspects, the present disclosure provides reactive polymers that comprise one or more hydrophilic polymer segments and have a plurality of hydrophilic polymer segment ends. At least a portion of the hydrophilic polymer segment ends have a reactive moiety are covalently linked to the hydrophilic polymer segment end. As detailed further below that reactive moieties are 3,4-substituted-2,5-pyrrolidinedione moieties in which the 3-carbon and the 4-carbon form part of at least one ring in addition to the 2,5-pyrrolidinedione ring.

In some embodiments, the reactive polymers comprise a single hydrophilic polymer segment, and the plurality of hydrophilic polymer segment ends comprise first and second ends of the single hydrophilic polymer segment.

In some embodiments, the reactive polymers comprise a plurality of hydrophilic polymer segments. For example, the reactive polymers may be reactive multi-arm polymers that comprise a plurality of polymer arms linked to a core region, where at least a portion of the polymer arms comprise a hydrophilic polymer segment that is covalently linked to the core region and a reactive moiety that is covalently linked to the hydrophilic polymer segment.

Reactive multi-arm polymers in accordance with the present disclosure include polymers comprising a core region and a plurality of polymer arms linked to the core region, at least a portion of the polymer arms comprising a hydrophilic polymer segment having first and second ends and a reactive moiety, the first end of the hydrophilic polymer segment covalently linked to the core region and the reactive moiety covalently linked to the second end of the hydrophilic polymer segment.

The reactive multi-arm polymers include polymers having two, three, four, five, six, seven, eight, nine, ten, eleven, twelve, fifteen, twenty, twenty-five or more arms.

In some embodiments, the reactive moiety may be covalently linked to the hydrophilic polymer segment through a hydrolysable ester group.

In some of these embodiments, the reactive moiety may be covalently linked to the hydrophilic polymer segment through a cyclic anhydride residue. In particular embodiments, at least a portion of the arms of the polymers may comprise a hydrophilic polymer segment linked to the core region, a cyclic anhydride residue that is covalently linked to the hydrophilic polymer segment, and a reactive moiety that is covalently linked to the cyclic anhydride residue. In particular embodiments, the arms of the polymers may comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Examples of cyclic anhydride residues include residues of glutaric anhydride, residues of succinic anhydride, residues of malonic anhydride, residues of adipic anhydride, and residues of diglycolic anhydride, among others.

In some embodiments, the cyclic anhydride residues are residues of iodine-containing cyclic anhydrides. Examples of iodine-containing cyclic anhydrides include cyclic anhydrides in which at least one ring carbon of the cyclic anhydride compound is substituted with iodine alone or an iodinated moiety. Examples include cyclic anhydride compounds in which at least one ring carbon is substituted with an iodinated moiety that comprises an iodinated aromatic group. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.). The iodinated aromatic groups may be directly linked to the ring carbon or may be linked to the ring carbon through any suitable linking moiety, which may be selected, for example, from an alkyl group, ether group, ester group, amide group, amine group, or carbonate group, or a combination thereof, among others.

A few specific examples of iodine-containing cyclic anhydrides for use in the present disclosure include the following iodine-containing glutaric anhydride compounds, among others: 4-(2,3,5-triiodophenyl)tetrahydropyran-2,6-dione, CAS #2357909-35-2,

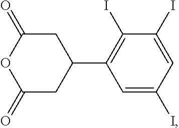 [SMILES: O=C1CC(c2cc(I)cc(I)c2I)CC(=O)O1]

4-(4-iodophenyl)tetrahydropyran-2,6-dione, CAS #2354237-72-0,

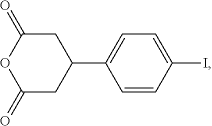 [SMILES: O=C1CC(c2ccc(I)cc2)CC(=O)O1]

4-((4-iodophenyl)methyl), tetrahydropyran-2,6-dione, CAS #2354625-91-3,

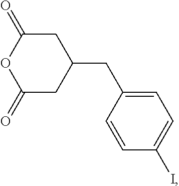 [SMILES: O=C1CC(Cc2ccc(I)cc2)CC(=O)O1]

3-(4-iodophenyl)tetrahydrofuran-2,5-dione, CAS #2354046-55-0,

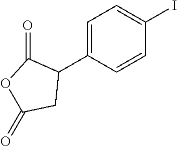 [SMILES: O=C1CC(c2ccc(I)cc2)C(=O)O1]

and 3-((4-iodophenyl)methyl)tetrahydrofuran-2,5-dione, CAS #2352978-66-4,

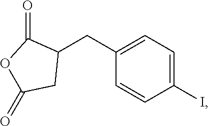 [SMILES: O=C1CC(Cc2ccc(I)cc2)C(=O)O1]

among others.

The above-described cyclic anhydrides, among others, may be reacted with a precursor polymer that comprises a core region and arms comprising hydroxy-terminated hydrophilic polymer segments extending from the core region, under ring opening conditions, to form a carboxylic-acid-terminated precursor polymer comprising arms that comprise a carboxylic acid end group that is linked to a hydrophilic polymer segment through a hydrolysable ester group. A reactive moiety may then be added to the carboxylic-acid-terminated precursor polymer.

In various embodiments an N-hydroxy cyclic imide compound, specifically, a 3,4-substituted-1-hydroxy-2,5-pyrrolidinedione compound in which the 3-carbon and the 4-carbon form part of at least one ring (e.g., a single-ring structure or a multi-ring structure) in addition to the 2,5-pyrrolidinedione ring may be reacted with a carboxylic-acid-terminated precursor polymer, such as that described above, to form a polymer comprising a plurality of 3,4-substituted-2,5-pyrrolidinedione reactive moieties in which the 3-carbon and 4-carbon form part of at least one ring in addition to the 2,5-pyrrolidinedione ring. The 2,5-pyrrolidinedione ring nitrogen atom of each of the 3,4-substituted-2,5-pyrrolidinedione reactive moieties is linked to a hydrophilic polymer segment through a hydrolysable ester group.

In some embodiments an N-hydroxy cyclic imide compound, specifically, a compound of the following formula,

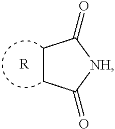

where R denotes a single-ring structure or a multi-ring structure, may be reacted with a carboxylic-acid-terminated precursor polymer, such as that described above, to form a polymer comprising arms that comprise a reactive moiety of the following formula,

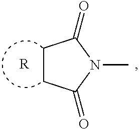

where R denotes a single-ring structure or a multi-ring structure, and where the reactive moiety may be linked to a hydrophilic polymer segment through a hydrolysable ester group.

Examples of single-ring and multi-ring structures include those that comprise a cyclopentane structure, a cyclopentene structure, a cyclohexane structure, a cyclohexene structure, a norbornane structure, a norbornene structure, a 7-oxonorbornane structure, or a 7-oxonorbornene structure, including substituted and unsubstituted cyclopentane structures, substituted and unsubstituted cyclopentene structures, substituted and unsubstituted cyclohexane structures, substituted and unsubstituted cyclohexene structures, substituted and unsubstituted norbornane structures, substituted and unsubstituted norbornene structures, substituted and unsubstituted 7-oxonorbornane structures, and substituted and unsubstituted 7-oxonorbornene structures.

The N-hydroxy cyclic imide compound may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent). Without wishing to be bound by theory, it is believed that the significant side reactions associated with impurity formation when using N-hydroxysuccinimide to form activated esters are avoided as a result of the ring-opening rearrangement process.

Specific examples of such N-hydroxy cyclic imide compounds include, for example,

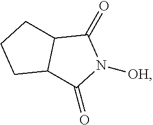 [SMILES: O=C1C2CCCC2C(=O)N1O]

N-hydroxy-1,2-cyclopentane dicarboxylic imide, CAS #412283-61-5,

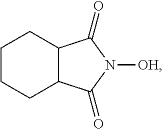 [SMILES: O=C1C2CCCCC2C(=O)N1O]

N-hydroxy-1,2-cyclohexane dicarboxylic imide, CAS #5426-10-8,

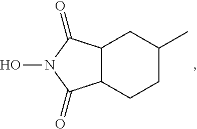

N-hydroxy-4-methyl-1,2-cyclohexane dicarboxylic imide, CAS #244140-93-0,

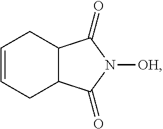 [SMILES: O=C1C2CC=CCC2C(=O)N1O]

N-hydroxy-4-Cyclohexene-1,2-dicarboxylic imide, CAS #7151-24-8,

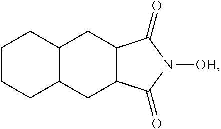 [SMILES: O=C1C2CC3CCCCC3CC2C(=O)N1O]

decahydro-2-hydroxy-1H-benz[f]isoindole-1,3(2H)-dione, CAS #137373-33-2,

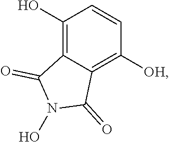 [SMILES: O=C1c2c(O)ccc(O)c2C(=O)N1O]

N-hydroxy-3,6-dihydroxy-1,2-benzene dicarboxylic imide CAS #216986-58-2,

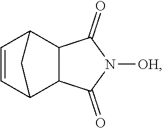 [SMILES: O=C1C2C3C=CC(C3)C2C(=O)N1O]

N-hydroxy-bicyclo[2.2.1]hept-5-ene-2,3-dicarboxylic imide, also known as N-Hydroxy-5-norbornene-2,3-dicarboxylic acid imide, or HONB), CAS #21715-90-2,

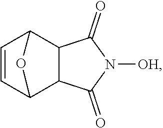 [SMILES: O=C1C2C3C=CC(O3)C2C(=O)N1O]

exo-N-hydroxy-7-oxabicyclo[2.2.1]hept-5-ene-2,3-dicarboximide, CAS #5596-17-8,

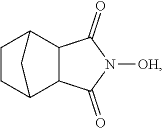 [SMILES: O=C1C2C3CCC(C3)C2C(=O)N1O]

N-hydroxy-norbornan-exo-2,3-dicarboximide, CAS #92619-32-4,

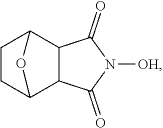 [SMILES: O=C1C2C3CCC(O3)C2C(=O)N1O]

N-hydroxy-1,4-epoxycyclohexane-2,3-dicarboxylic imide, CAS #88179-68-4,

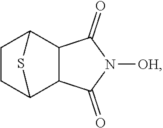 [SMILES: O=C1C2C3CCC(S3)C2C(=O)N1O]

N-hydroxy-1,4-epithiocyclohexane-2,3-dicarboxylic imide, CAS #327974-00-5,

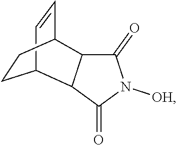 [SMILES: O=C1C2C3C=CC(CC3)C2C(=O)N1O]

3a,4,7,7a-tetrahydro-2-hydroxy-4,7-ethano-1H-isoindole-1,3(2H)-dione, CAS #396133-48-5, and

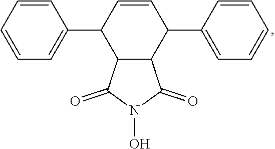 [SMILES: O=C1C2C(c3ccccc3)C=CC(c3ccccc3)C2C(=O)N1O]

3a,4,7,7a-tetrahydro-2-hydroxy-4,7-diphenyl-1H-isoindole-1,3(2H)-dione, CAS #1629567-11-8.

For example, in the particular case of N-Hydroxy-5-norbornene-2,3-dicarboxylic acid imide (HONB) as a 3,4-substituted-1-hydroxy-2,5-pyrrolidinedione compound, exemplary reactive end groups that can be formed include 5-norbornene-2,3-dicarboxylic acid imidyl malonate groups, 5-norbornene-2,3-dicarboxylic acid imidyl glutarate groups, 5-norbornene-2,3-dicarboxylic acid imidyl succinate groups, 5-norbornene-2,3-dicarboxylic acid imidyl adipate groups, and 5-norbornene-2,3-dicarboxylic acid imidyl diglycolate groups, among others.

Hydrophilic polymer segments for used in the reactive polymers of the present disclosure can be selected from any of a variety of synthetic, natural, or hybrid synthetic-natural hydrophilic polymer segments. Examples of hydrophilic polymer segments include those that are formed from one or more hydrophilic monomers selected from ethylene oxide, propylene oxide, N-vinyl pyrrolidone, oxazoline monomers (e.g., oxazoline and 2-alkyl-2-oxazolines, for instance, 2-(C₁-C₆ alkyl)-2-oxazolines (including various isomers), such as 2-methyl-2-oxazoline, 2-ethyl-2-oxazoline, 2-propyl-2-oxazoline, 2-isopropyl-2-oxazoline, 2-n-butyl-2-oxazoline, 2-hexyl-2-oxazoline, etc.), 2-phenyl-2-oxazoline, N-isopropylacrylamide, amino acids and sugars. In some embodiments, the hydrophilic polymer segments are iodine-containing hydrophilic polymer segments.

Hydrophilic polymer segments may be selected, for example, from the following polymer segments: polyether segments including poly(alkylene oxide) segments such as poly(ethylene oxide) (PEO) (also referred to as polyethylene glycol or PEG) segments, poly(propylene oxide) segments, poly(ethylene oxide-co-propylene oxide) segments, poly(N-vinyl pyrrolidone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, protein segments or polysaccharide segments. Polymer segments for use in the multi-arm polymers of the present disclosure typically contain between 10 and 1000 monomer units.

As previously noted, in the case of reactive multi-arm polymers, the polymer arms extend from a core region. In certain of these embodiments, the core region comprises a residue of a polyol comprising two or more hydroxyl groups, which is used to form the polymer arms. In certain beneficial embodiments, the core region comprises a residue of a polyol that contains two, three, four, five, six, seven, eight, nine, ten, eleven, twelve, fifteen, twenty, twenty-five or more hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others. Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 10 to 1000 units in length.

In some embodiments, the polyol is an iodine-containing polyol. Examples of iodine-containing polyols include polyols in which at least one carbon atom of the polyol is substituted with iodine alone or with an iodinated moiety. Examples include polyols in which at least one carbon is substituted with an iodinated moiety that comprises an iodinated aromatic group. Examples of iodinated aromatic groups include those described above. The iodinated aromatic groups may be directly linked to the polyol carbon or may be linked to the polyol carbon through any suitable linking moiety, which may be selected, for example, from an alkyl group, ether group, ester group, amide group, amine group, carbonate group, or a combination thereof, among others.

As specific examples, commercially available 1,3,5 triiodo-2,4,6-trishydroxymethylbenzene (CAS #178814-33-0),

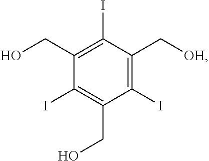 [SMILES: OCc1c(I)c(CO)c(I)c(CO)c1I]

can be used directly as an initiator for the polymerization of a 3-arm polymer with 3 iodine atoms at the core, commercially available iodixanol (CAS #92339-11-2),

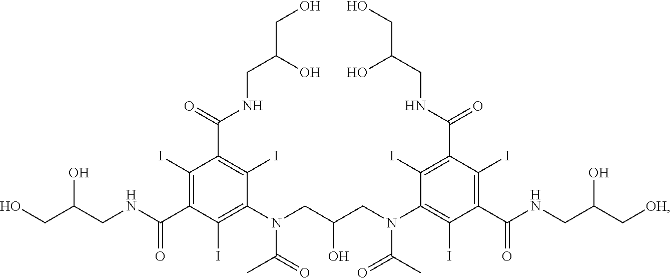 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

can be used directly as an initiator for the polymerization of an 8-arm polymer with 6 iodine atoms at the core, iohexol (CAS #66108-95-0)

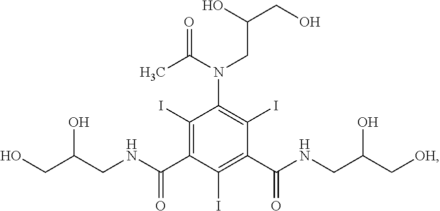 [SMILES: CC(=O)N(CC(O)CO)c1c(I)c(C(=O)NCC(O)CO)c(I)c(C(=O)NCC(O)CO)c1I]

can be used directly as an initiator for the polymerization of a 6-arm polymer with 3 iodine atoms at the core, iohexol impurity J (CAS #76801-93-9),

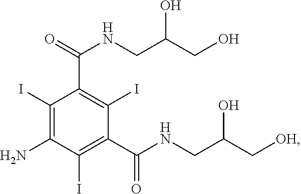 [SMILES: Nc1c(I)c(C(=O)NCC(O)CO)c(I)c(C(=O)NCC(O)CO)c1I]

can be used directly as an initiator for the polymerization of a 4-arm polymer with 3 iodine atoms at the core, and so forth.

In other embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

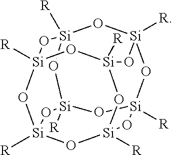

In the present disclosure, the R groups comprise the polymer arms described herein.

Reactive polymers in accordance with the present disclosure can be formed from hydroxy-terminated precursor polymers having one or more hydrophilic polymer segments with one or more hydroxyl end groups.

In particular embodiments, terminal hydroxyl groups of the one or more hydrophilic polymer segments are reacted with a cyclic anhydride (e.g., glutaric anhydride, succinic anhydride, malonic anhydride, adipic anhydride, diglycolic anhydride, etc.) to form a reaction product that comprises carboxylic acid groups that are linked to the one or more hydrophilic segments through a hydrolysable ester group. This reaction product is then reacted with a 3,4-substituted-1-hydroxy-2,5-pyrrolidinedione compound in which the 3-carbon and the 4-carbon form part of at least one ring in addition to the 2,5-pyrrolidinedione ring in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to yield a polymer that comprises a plurality of reactive 3,4-substituted-2,5-pyrrolidinedione moieties in which the 3-carbon and 4-carbon form part of at least one ring in addition to the 2,5-pyrrolidinedione ring, wherein the 2,5-pyrrolidinedione ring nitrogen atoms of the 3,4-substituted-2,5-pyrrolidinedione moieties are linked to the hydrophilic polymer segments through a hydrolysable ester group, such as a malonate group, a glutarate group, a succinate group, an adipate group, or a diglycolate group.

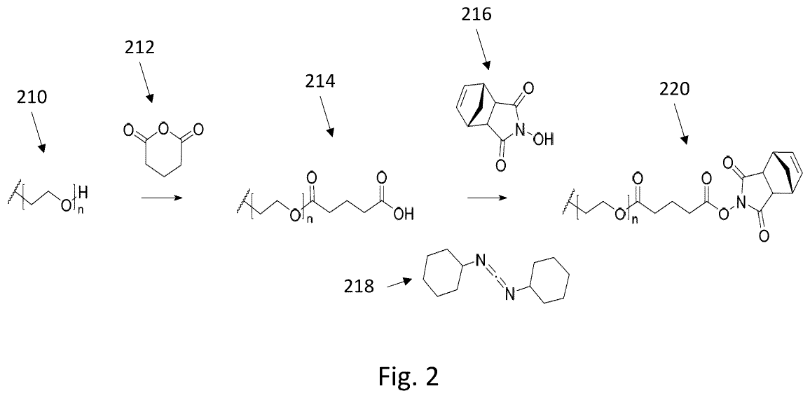

In a particular embodiment shown in FIG. 2, a multi-arm polymer 210 comprising multiple hydroxyl-terminated poly(ethylene glycol) arms (e.g., where n is two, three, four, five, six, seven, eight, nine, ten, eleven, twelve, fifteen, twenty, twenty-five or more) is reacted with glutaric anhydride 212 to form a multi-arm polymer product 214 that comprises multiple glutaric-acid-ester-terminated poly(ethylene glycol) arms, in which a carboxylic acid group is linked to the poly(ethylene glycol) segment through a hydrolysable ester group. This multi-arm polymer product 214 is then reacted with N-hydroxy-5-norbornene-2,3-dicarboxylic acid imide (HONB) 216 in the presence of a N,N′-dicyclohexylcarbodiimide coupling agent 218 to yield a reactive multi-arm polymer 220 in which the arms comprise poly(ethylene glycol) segments with reactive 5-norbornene-2,3-dicarboxylic acid imidyl glutarate end groups.

In some aspects, the present disclosure provides a hydrogel that comprises a crosslinked reaction product of (a) a reactive polymer as described herein and (b) a compound having a plurality of reactive nucleophilic moieties (e.g. amine moieties and/or thiol moieties, among others).

In particular embodiments, the compound having a plurality of reactive nucleophilic moieties is a polyamino compound. In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamino compounds which may be used as the polyamino compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

In some embodiments, the polyamino compounds may be substituted with one or more radiopaque atoms such as iodine or bromine.

In some embodiments, the crosslinked reaction products of the present disclosure are visible under fluoroscopy. Such crosslinked products may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, systems are provided that are configured to deliver (a) a polyamino compound (although a polyamino compound is described herein, it will be appreciated that other compounds having a plurality of reactive nucleophilic moieties may be employed) and (b) a reactive polymer as described herein. The polyamino compound and the reactive polymer are combined under conditions such that the amino groups of the polyamino compound and the reactive moieties of the reactive polymer crosslink with one another. In certain embodiments, those conditions comprise an environment having a basic pH, for example, a pH ranging from about 9 to about 11. Such systems can be used to form crosslinked hydrogels, either in vivo or ex vivo.

In some aspects of the present disclosure, a system is provided that comprises (a) a first composition that comprises a polyamino compound, for example, as described herein (although a polyamino compound is described herein, it will be appreciated that other compounds having a plurality of reactive nucleophilic moieties may be employed) and (b) a second composition that comprises a reactive polymer as described herein.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the polyamino compound is initially combined with the reactive polymer at an acidic pH at which crosslinking between the reactive moieties of the reactive polymer and the amino groups of the polyamino compound is suppressed. Then, when crosslinking is desired, a pH of the mixture of the polyamino compound and the reactive polymer is changed from an acidic pH to a basic pH, leading to crosslinking between same, thereby forming the crosslinked product.

In particular embodiments, the system comprises (a) a first composition that comprises a polyamino compound as described hereinabove, (b) a second composition that comprises a reactive polymer as described hereinabove, and (c) a third composition, specifically, an accelerant composition, that contains an accelerant that is configured to accelerate a crosslinking reaction between the polyamino compound and the reactive polymer.

The first composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the polyamino compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the reactive polymer. The first composition may then be mixed with the second composition to provide a prepared fluid composition that is buffered to an acidic pH and comprises the polyamino compound and the reactive polymer. In a particular example, a syringe may be provided that contains a first fluid composition comprising the polyamino compound that is buffered to an acidic pH, and a vial may be provided that comprises a dry composition (e.g., a powder) that comprises the reactive polymer. The syringe may then be used to inject the first fluid composition into the vial containing the reactive polymer to form a prepared fluid composition that contains the polyamino compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

A prepared fluid composition that is buffered to an acidic pH and comprises the polyamino compound and the reactive polymer as described above, and a fluid accelerant composition that is buffered to basic pH as described above, may be combined form crosslinked hydrogels, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a polyamino compound as described above and a second reservoir that contains a second composition that comprises a reactive polymer that comprises a plurality of reactive moieties that are reactive with the amino moieties of the polyamino compound as described above.

In some embodiments, the system may include a delivery device that comprises a first reservoir that contains a first composition that comprises the polyamino compound and the reactive polymer and is buffered to an acidic pH, such as the prepared fluid composition previously described, and a second reservoir that contains second composition, such as the fluid accelerant composition previously described. In either case, during operation, the first composition and second composition are dispensed from the first and second reservoirs and combined, whereupon the polyamino compound and the reactive polymer and crosslink with one another to form a crosslinked hydrogel.

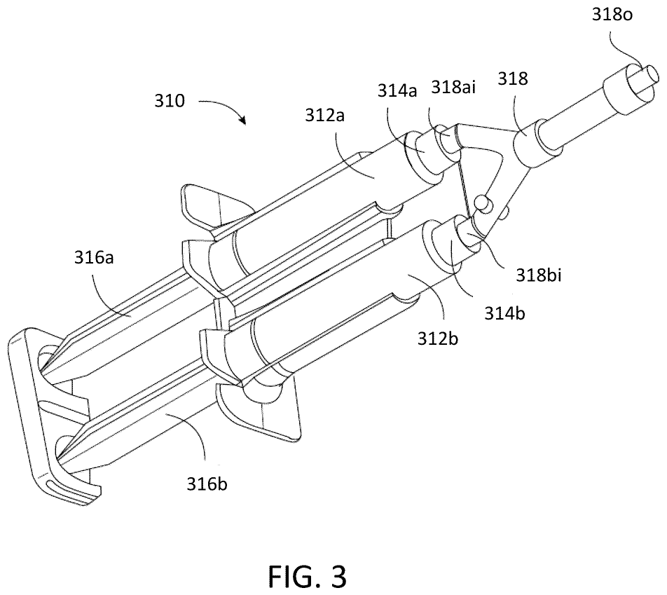

In particular embodiments, and with reference to FIG. 3, the system may include a delivery device 310 that comprises a double-barrel syringe, which includes first barrel 312a having a first barrel outlet 314a, which first barrel contains the first composition, a first plunger 316a that is movable in the first barrel 312a, a second barrel 312b having a second barrel outlet 314b, which second barrel 312b contains the second composition, and a second plunger 316b that is movable in the second barrel 312b. In some embodiments, the device 310 may further comprise a mixing section 318 having a first mixing section inlet 318ai in fluid communication with the first barrel outlet 314a, a second mixing section inlet 318bi in fluid communication with the second barrel outlet, and a mixing section outlet 318o.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), crushing, milling, pounding, or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

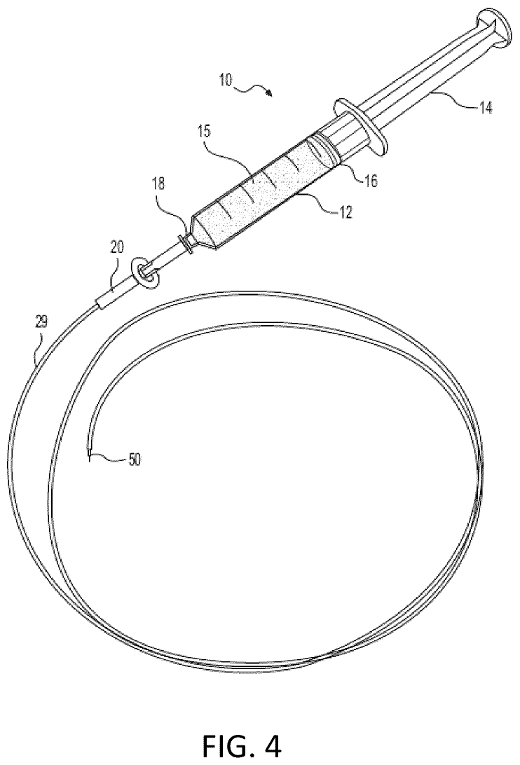

FIG. 4 illustrates a syringe 10 providing a reservoir for a crosslinked hydrogel compositions as discussed above (e.g., a suspension of crosslinked hydrogel particles). The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or the crosslinked hydrogel compositions can be injected as a carrier of therapeutic agents in the treatment of diseases and cancers and the repair and regeneration of tissue, among other uses.

After administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure also include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A reactive polymer comprising one or more hydrophilic polymer segments having a plurality of hydrophilic polymer segment ends and a plurality of reactive moieties covalently linked to at least a portion of the hydrophilic polymer segment ends, wherein the reactive moieties are 3,4-substituted-2,5-pyrrolidinedione moieties in which the 3-carbon and the 4-carbon form part of at least one ring in addition to the 2,5-pyrrolidinedione ring and wherein the 2,5-pyrrolidinedione ring nitrogen atom of each of the 3,4-substituted-2,5-pyrrolidinedione moieties is linked to one of the hydrophilic polymer segment ends.

2. The reactive polymer of claim 1, wherein the 2,5-pyrrolidinedione ring nitrogen atom is linked to the one of the hydrophilic polymer segment ends through a hydrolysable ester group.

3. The reactive polymer of claim 1, wherein the one or more hydrophilic polymer segments comprise a single hydrophilic polymer segment, and the plurality of hydrophilic polymer segment ends comprise first and second ends of the single hydrophilic polymer segment.

4. The reactive polymer of claim 1, comprising a plurality of polymer arms linked to a core region, at least a portion the polymer arms each comprising one of the plurality hydrophilic polymer segments and one of the plurality of reactive moieties.

5. The reactive polymer of claim 4, wherein a first end of the one of the plurality of hydrophilic polymer segments is covalently linked to the core region, a second end of the one of the plurality of hydrophilic polymer segment is covalently linked to a first end of a cyclic anhydride residue, and the one of the plurality of reactive moieties is covalently linked to a second end of the cyclic anhydride residue.

6. The reactive polymer of claim 1, wherein the hydrophilic polymer segment comprises one or more monomer residues selected from ethylene oxide, propylene oxide, N-vinyl pyrrolidone and oxazoline monomer residues.

7. The reactive polymer of claim 1, wherein each of the reactive moieties comprise the following formula, 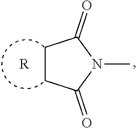
where R denotes a single-ring or a multi-ring structure.

8. The reactive polymer of claim 7, wherein R denotes a cyclopentane structure, a cyclopentene structure, a cyclohexane structure, a cyclohexene structure, a norbornane structure, a norbornene structure, a 7-oxonorbornane structure, or a 7-oxonorbornene structure.

9. A method of treatment comprising administering to a subject a mixture that comprises (a) a nucleophilic compound and (b) a reactive polymer in accordance with claim 1, wherein the mixture is administered under conditions such that the nucleophilic compound and the reactive polymer crosslink after administration.

10. A system for forming a hydrogel composition that comprises (a) a nucleophilic compound and (b) a reactive polymer comprising one or more hydrophilic polymer segments having a plurality of hydrophilic polymer segment ends and a plurality of reactive moieties covalently linked to at least a portion of the hydrophilic polymer segment ends, wherein the reactive moieties are 3,4-substituted-2,5-pyrrolidinedione moieties in which the 3-carbon and the 4-carbon form part of at least one ring in addition to the 2,5-pyrrolidinedione ring and wherein the 2,5-pyrrolidinedione ring nitrogen atom of each of the 3,4-substituted-2,5-pyrrolidinedione moieties is linked to one of the hydrophilic polymer segment ends.

11. The system of claim 10, wherein the nucleophilic compound is a polyamino compound.

12. The system of claim 10, wherein the system comprises a first composition that comprises the nucleophilic compound and a second composition that comprises the reactive polymer.

13. The system of claim 12, further comprising an accelerant composition.

14. The system of claim 10, further comprising a delivery device.

15. A synthesis method comprising condensing (a) a 3,4-substituted-1-hydroxy-2,5-pyrrolidinedione compound in which the 3-carbon and the 4-carbon form part of at least one ring in addition to the 2,5-pyrrolidinedione ring with (b) a precursor polymer comprising one or more hydrophilic polymer segments having a plurality of hydrophilic polymer segment ends that are terminated in carboxylic acid groups, thereby forming a reactive polymer comprising a plurality of reactive moieties covalently linked to the hydrophilic polymer segment ends, wherein each of the reactive moieties is a 3,4-substituted-2,5-pyrrolidinedione moiety in which the 3-carbon and the 4-carbon form part of at least one ring in addition to the 2,5-pyrrolidinedione ring, and wherein the 2,5-pyrrolidinedione ring nitrogen atom of the 3,4-substituted-2,5-pyrrolidinedione moiety is linked to one of the hydrophilic polymer segment ends.

16. The synthesis method of claim 15, wherein the precursor polymer is formed by reacting an acid anhydride with a polymer comprising one or more hydrophilic polymer segments having a plurality of hydrophilic polymer segment ends that are terminated in hydroxyl groups.

17. The synthesis method of claim 15, wherein the one or more hydrophilic polymer segments comprise a single hydrophilic polymer segment, and the plurality of hydrophilic polymer segment ends comprise first and second ends of the single hydrophilic polymer segment.

18. The synthesis method of claim 15, wherein the precursor polymer comprises a plurality of polymer arms linked to a core region, at least a portion the polymer arms each comprising one of the plurality hydrophilic polymer segments and one of the plurality of reactive moieties.

19. The synthesis method of claim 15, wherein the 3,4-substituted-1-hydroxy-2,5-pyrrolidinedione compound is a compound of the following formula, 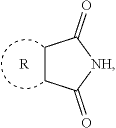
where R denotes a single-ring or a multi-ring structure.

20. The synthesis method of claim 19, wherein R denotes a cyclopentane structure, a cyclopentene structure, a cyclohexane structure, a cyclohexene structure, a norbornane structure, a norbornene structure, a 7-oxonorbornane structure or a 7-oxonorbornene structure.
